(19) United States
(12) Patent Application Publication
(10) Pub. No.: US 2014/0194529 A1
(43) Pub. Date: Jul. 10, 2014

(54) USAGE OF NATURAL COMPOUND FOR TREATING INFLAMMATION AND CANCER

(71) Applicant: Hong Kong Baptist University, Hong Kong (HK)

(72) Inventors: Hongxi XU, HK (HK); Dandan ZHANG, HK (HK); Hong ZHANG, HK (HK); Kaixian CHEN, HK (HK); Zhaoxiang BIAN, HK (HK); Chengyuan LIN, HK (HK); Dajian YANG, HK (HK); Shilin CHEN, HK (HK); Aiping LU, HK (HK); Albert Sun Chi CHAN, HK (HK)

(73) Assignee: Hong Kong Baptist University, Hong Kong (HK)

(21) Appl. No.: 14/149,820

(22) Filed: Jan. 8, 2014

Related U.S. Application Data
(60) Provisional application No. 61/750,887, filed on Jan. 10, 2013.

(51) Int. Cl. C07C 49/83

(52) U.S. Cl. CPC C07C 49/83
USPC 514/681; 568/327

(57) ABSTRACT

This invention relates to the use of a compound from natural sources for therapeutic uses. More particularly, it relates to a compound, Garcimultiflorone E, naturally occurring in the plant of Garcinia esculenta Y. H. Li which has potent anti-inflammation and anti-cancer effect.

### CROSS-REFERENCE TO RELATED APPLICATIONS

This application claims the benefit of U.S. Provisional Patent Application Ser. No. 61/750,887 filed on 10 Jan. 2013, disclosure of which is incorporated herein by reference in its entirety.

### FIELD OF INVENTION

This invention is in the field of pharmaceuticals and chemical industries. In particular, this invention relates to the use of a compound from natural sources for therapeutic uses. More particularly, it relates to a compound, Garcimultiflorone E, naturally occurring in the plant of Garcinia esculenta Y. H. Li which inhibits nitric oxide production and exhibits potent anti-inflammation and anti-cancer effect. The present invention provides a novel anti-cancer and anti-inflammation composition and method of using the same for cancer treatment.

### BACKGROUND OF INVENTION

The normal inflammatory process represent a protective mechanism of the body initially, excessive and chronic inflammation results in damage of the cells and tissues. The merging evidences support the hypothesis that inflammation plays a key role in various chronic pathological conditions including auto-immundisorder, hypertension, atherosclerosis, stroke, metabolic diseases, cancer, and neurodegenerative diseases. Nitric oxide (NO) chemically belongs to free radical which is synthesized from L-arginine by nitric oxide synthase (NOS). Constitutive isoform of NOS (cNOS) includes eNOS and nNOS, which generate abnormal concentration of NO and mediate various physiological functions. However, the inducible isoform of the NOS (iNOS) in majority cases only express upon the stimulation by inflammatory factors, and can produce excess amount of NO which promoting inflammatory reactions and may have pathological consequences. It is notable that various inflammatory stimuli can activate distinct signaling pathways that converge to trigger the expression of iNOS.

Macrophages play a central role in inflammatory response and serve as an essential interface between innate and adaptive immunity. The massive amounts of NO produced in response to bacterial Lipopolysaccharide (LPS) or cytokines play an important role in the inflammatory condition. Thus, RAW 264.7 macrophages provide us with an excellent model for anti-inflammatory drug screening.

Natural products have played a significant role in anti-inflammatory drug discovery and development. Drug designs that decrease NO production have a therapeutic effect in the treatment of septic shock, as well as many other inflammatory and immune disorder.

The objective of the invention is to provide a compound useful for anti-inflammatory and anti-cancer applications, and to provide method for anti-inflammatory and anti-cancer treatment comprises the use thereof.

Citation or identification of any reference in this section or any other section of this application shall not be construed as an admission that such reference is available as prior art for the present application.

### SUMMARY OF INVENTION

Accordingly, the objective of this invention is to provide a compound from a natural source that exhibits potent anti-inflammation and anti-cancer effect. A novel anti-cancer and anti-inflammatory composition and method of using the same for treatment cancer and inflammatory are provided.

In accordance with one aspect of the present invention, there is provided a compound, Garcimultiflorone E (GE, FIG. 1), naturally occurring in the plant of Garcinia esculenta Y. H. Li which inhibits nitric oxide production and exhibits potent anti-inflammation effect, for use as an active compound in drugs for inflammation treatment.

In accordance with another aspect of the present invention, there is provided a compound, Garcimultiflorone E, naturally occurring in the plant of Garcinia esculenta Y. H. Li, which inhibits nitric oxide production and exhibits potent anti-cancer effect, for use as an active compound in drugs for cancer treatment.

Those skilled in the art will appreciate that the invention described herein is susceptible to variations and modifications other than those specifically described.

The invention includes all such variation and modifications. The invention also includes all of the steps and features referred to or indicated in the specification, individually or collectively and any and all combinations or any two or more of the steps or features.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows the structure of Garcimultiflorone E.

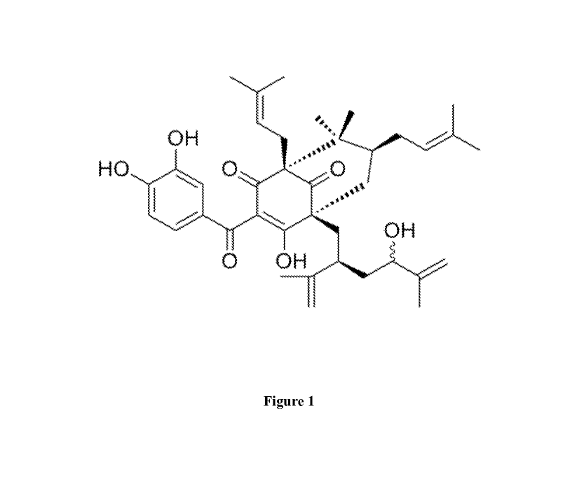

### DETAILED DESCRIPTION OF INVENTION

The present invention is not to be limited in scope by any of the specific embodiments described herein. The following embodiments are presented for exemplification only.

Garcinia esculenta Y. H. Li belongs to Garcinia genus of plants. It is one of endemic plants in China, which mainly grew up in the valley and areas above 860-1650 meters sea level, and has not been artificially cultivated. Matured fruit is edible.

Reagents

Murine recombinant IFN-γ was purchased from Millpore, Lipopolysaccharide, dimethyl sulfoxide, Indometacin were obtained from Sigma. RPMI-1640 was purchased from Gibco. Fetal bovine serum (FBS) was purchased from Hyclone.

Cell Culture

RAW 264.7 cells were obtained from the American Tissue Culture Collection (ATCC). Liver cancer line SMMC-7721 and HepG2, breast cancer lines including MCF-7 and MDA-MB-231 were purchased from cell-bank of Chinese Academy of Sciences, China. These cells were maintained in complete RPMI 1640 medium supplemented with 10% FBS at 37° C. in a humidified 5% CO2 atmosphere.

Assay for NO Accumulation

RAW264.7 were maintained in RPMI 1640 medium supplemented with 10% FBS at 37° C. in a humidified 5% CO2 atmosphere. Cells were plated in a 96-well plate (5×103 cells/well) and allowed to attach overnight. The cells were then stimulated with 10 U/ml of IFN-γ and 100 ng/ml of LPS for 24 hours in the presence or absence of GE with final concentration of 50, 25, 12.5, 6.25, 3.125 μg/mL, and Indometacin as positive drug.

Assay for iNOS Function (Griess Reaction)

RAW264.7 were maintained in RPMI 1640 medium supplemented with 10% FBS at 37° C. in a humidified 5% CO2 atmosphere. Cells were plated in a 96-well plate (5×103 cells/well) and allowed to attach overnight. For stimulation, the medium was replaced with fresh FBS-free RPMI 1640 for 10 hours, and the cells were then stimulated with 10 U/ml of IFN-γ and 100 ng/ml of LPS for 24 hours in the presence or absence of extractions or fractions.

iNOS function was analyzed with Griess reaction by assay of nitrite concentration in the culture supernatant. Briefly, 100 μl/well of sample was incubated with equal volume of Griess solution (1% sulfanilamide in 5% phosphoric acid+1% α-naphthylamine in distilled water) at room temperature for 10 minutes. The absorbance was evaluated at 540 nm using a calibration curve with a sodium nitrite standard. Percent inhibition was expressed as [1−(NO level of sample/NO level of vehicle treated−control)]×100.

Assay for Cell Cytotoxicity

SMMC-7721 and HepG2, MCF-7 and MDA-MB-231 were seeded into 96-well plates (3×103 cells/well) and allowed to attach overnight. GE were added in final concentration of 50, 25, 12.5, 6.25, 3.125 μg/mL and continually cultured for 72 hours. Taxol as positive drug. Each well was incubated with 20 μl MTT (5 mg/ml in phosphate-buffered saline) for 4 hours at 37° C. Volume of 150 μl DMSO was added after the medium in well discarded and reacted for 10 minutes. Absorbance was measured at 490 nm. The optical density of formazan formed in control (untreated) cells was taken as 100% of viability. Mean value of each concentration (n=3 wells) was obtained.

Statistical Analysis

Results are presented as means±SD of three independent experiments. Student's test was used for each significant effect of treatment. Comparisons between multiple groups were performed with one-way ANOVA test. The threshold of significance was set at P<0.05.

Results

Concentration-Dependent Inhibition of iNOS-NO Production by Garcimultiflorone E.

To demonstrate the effect of Garcimultiflorone E on iNOS-induced NO generation in RAW 264.7 cells, LPS/IFN-γ double stimulation model to observe the nitrite accumulation is used. 24 hours inflammatory factors treatment trigger folds increase of nitrite concentration, as shown in Table 1. Application of Garcimultiflorone E dose-dependently attenuates NO production at IC50 of 5.78 μM.

TABLE 1
The inhibitory effect on NO accumulation of GE

| Sample | IC50 (μg/mL) | IC50 (μM) |
|---|---|---|
| GE | 3.57 | 5.78 |

Note:
The inhibitory effect on NO accumulation of Indometacin (1 μg/mL) is 46.51%.

Cell Cytotoxicity Valuation of Garcimultiflorone E

Cytotoxicity of the Garcimultiflorone E is shown in cell viability assay by using MTT method. As illustrated in Table 2, Garcimultiflorone E reduces the proliferation of SMMC-7721, HepG2 and MCF-7 at IC50 of 11.94, 13.16 and 9.63 μM, but has no effect on MDA-MB-231 cell line.

TABLE 2
The cell cytotoxicity effect of GE

| | IC50 (μM) | | | |
|---|---|---|---|---|
| Compound | SMMC-7721 | HepG2 | MCF-7 | MDA-MB-231 |
| GE | 11.94 | 13.16 | 9.63 | −15 |
| Taxol | 0.15 | 4.4 * 10−4 | 3.0 * 10−3 | 5.0 |

### CONCLUSION

Garcimultiflorone E inhibits proliferation of breast cancer lines and liver cancer lines at about 10 μM. What's more important that garcimultiflorone E also reduces the NO accumulation in cell inflammatory. The present invention provides a composition for anti-cancer and anti-inflammatory treatment comprises garcimultiflorone E and method of using the same for cancer and inflammatory treatment.

### INDUSTRIAL APPLICATION

The present invention discloses the use of a compound from natural sources for therapeutic uses. More particularly, it relates to a compound, Garcimultiflorone E, naturally occurring in the plant of Garcinia esculenta Y. H. Li, which inhibits nitric oxide production and exhibits potent anti-inflammation and anti-cancer effect.

If desired, the different functions discussed herein may be performed in a different order and/or concurrently with each other. Furthermore, if desired, one or more of the above-described functions may be optional or may be combined.

While the foregoing invention has been described with respect to various embodiments and examples, it is understood that other embodiments are within the scope of the present invention as expressed in the following claims and their equivalents. Moreover, the above specific examples are to be construed as merely illustrative, and not limitative of the reminder of the disclosure in any way whatsoever. Without further elaboration, it is believed that one skilled in the art can, based on the description herein, utilize the present invention to its fullest extend. All publications recited herein are hereby incorporated by reference in their entirety.

What we claim:

1. A composition for treating cancer comprising an effective amount of a compound with the chemical structure of

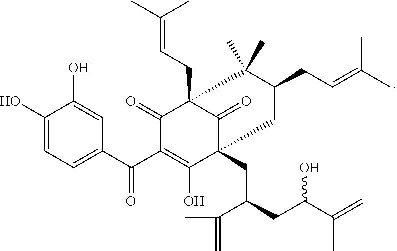

2. The composition according to claim 1 wherein said compound comprising Garcimultiflorone E.

3. The composition according to claim 1 wherein said compound is extracted from a natural plant.

4. The composition according to claim 3 wherein the natural plant is from a genus Garcinia.

5. The composition according to claim 4 wherein the genus Garcinia comprising a subspecies of Garcinia esculenta Y. H. Li.

6. The composition according to claim 1 wherein the cancer comprising breast cancer and liver cancer.

7. A composition for treating inflammation comprising an effective amount of a compound with the chemical structure of

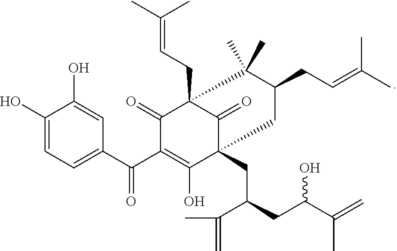

8. The composition according to claim 7 wherein said compound comprising Garcimultiflorone E.

9. The composition according to claim 7 wherein said compound is extracted from a natural plant.

10. The composition according to claim 9 wherein the natural plant is from a genus Garcinia.

11. The composition according to claim 10 wherein the genus Garcinia comprising a subspecies of Garcinia esculenta Y. H. Li.

12. A use of the composition according to claim 1 for manufacture of a medicament for treatment of cancer.

13. A use of the composition according to claim 7 for manufacture of a medicament for treatment of inflammation.

14. A method of treating cancer comprising administering a composition of claim 1, wherein the cancer comprising breast or liver cancer.

15. A method of treating inflammation comprising administering a composition of claim 7.

16. The method according to claim 14 wherein the effective amount is about 10 μM.

17. The method according to claim 15 wherein the effective amount is about 10 μM.
